(19) United States
(12) Patent Application Publication
(10) Pub. No.: US 2004/0058899 A1
(43) Pub. Date: Mar. 25, 2004

(54) Use of epothilones and analogs in conjunction with ophthalmic surgery

(76) Inventor: Peter G. Klimko, Fort Worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/275,463

(22) PCT Filed: Sep. 25, 2002
(86) PCT No.: PCT/US02/30315

(51) Int. Cl. A61K 31/427; A61K 31/365

(52) U.S. Cl. 514/183; 514/365; 514/450

(57) ABSTRACT

A method of use is disclosed for preventing or retarding posterior capsular opacification or scar formation following ophthalmic surgery by treatment of the surgical site with epothilones and analogs thereof.

### FIELD OF THE INVENTION

The present invention is directed to the use of compositions containing epothilones and their analogs in conjunction with ophthalmic surgery. These compositions may be used to prevent or treat posterior capsular opacification following extracapsular extraction of a cataractous lens with or without artificial intraocular lens implantation. They may also be used to prevent or retard fistula closure resulted from glaucoma filtration surgery (GFS).

### BACKGROUND OF THE INVENTION

1. Cataracts

Cataract extraction, a common surgical procedure, is performed on a patient when the natural crystallin lens of the eye becomes, for a variety of reasons, clouded or cataractous. Typically, the procedure involves i) making an incision in the anterior chamber of the eye in order to gain access to the lens capsular bag, ii) disintegration and removal of the defective lens, and iii) implantation of an artificial intraocular lens (IOL). A portion of the anterior membrane of the lens capsular bag is removed in order to allow implantation of the IOL. The capsular bag is normally cleaned by the ophthalmic surgeon in order to remove residual lens epithelial cells and other tissue debris. However it is difficult to remove all of the remnant tissue; consequently the residual lens epithelial cells frequently proliferate and cause eventual opacification of the IOL, leading to vision impairment in the patient. This result is termed posterior capsular opacification (PCO) or secondary cataract.

Treatment of PCO after the fact typically involves the destruction of the undesireable tissue using a laser. However, this procedure can have significant sight-threatening complications, including without limitation damage to the IOL, injury or destruction of surrounding healthy tissue, and renewed PCO development. Therefore, it is generally thought to be preferable to prevent PCO at the time of the cataract surgery. Administration of cytotoxic and cytostatic drugs to the affected site during surgery such as 5-fluorouracil [Ruitz el. al., Ophthalmic Res., volume 22, p. 201 (1990)], mitomycin C (ref), colchicine (ref), and taxol (Mannson el. al., U.S. Pat. No. 5,576,345) have been suggested in the art for PCO prevention. While these therapies can be helpful, serious side effects such as optic nerve and corneal epithelium toxicities sometimes occur. Thus, there remains a need for an efficacious therapy for the prevention and treatment of PCO incident to cataract extraction and IOL implantation.

2. Glaucoma

Glaucoma is a progressive disease which leads to optic nerve damage and, ultimately, total loss of vision. The causes of this disease have been the subject of extensive studies for many years, but are still not fully understood. The principal symptom of and/or risk factor for the disease is elevated intraocular pressure or ocular hypertension due to excess aqueous humor in the anterior chamber of the eye.

The causes of aqueous humor accumulation in the anterior chamber are not fully understood. It is known that elevated intraocular pressure (“IOP”) can be at least partially controlled by administering drugs which either reduce the production of aqueous humor within the eye, such as beta-blockers and carbonic anhydrase inhibitors, or increase the outflow of aqueous humor from the eye, such as miotics and sympathomimetics.

Most types of drugs conventionally used to treat glaucoma have potentially serious side effects. Miotics such as pilocarpine can cause blurring of vision and other visual side effects, which may lead either to decreased patient compliance or to Us termination of therapy. Systemically administered carbonic anhydrase inhibitors (CAIs) can also cause serious side effects such as nausea, dyspepsia, fatigue, and metabolic acidosis, which can affect patient compliance and/or necessitate the termination of treatment. Another type of drug, beta-blockers, have increasingly become associated with serious pulmonary side effects attributable to their effects on beta-2 receptors in pulmonary tissue. Sympathomimetics, on the other hand, may cause tachycardia, arrhythmia and hypertension.

Recently introduced drugs to treat glaucoma, such as topical CAIs and prostaglandin analogues, have addressed some of these issues, but there are still patients whose pressures are not adequately controlled by drug therapy alone. In many of these cases there is a severe blockage of the normal passages for the outflow of aqueous humor. These patients may require surgery to restore the normal outflow of aqueous humor and thereby normalize their IOPs. The outflow of aqueous humor can be improved by various intraocular surgical procedures known to those skilled in the art, including without limitation trabeculectomy, posterior lip sclerectomy, and trephine and thermal sclerostomy. These surgical procedures are collectively known as glaucoma filtration surgery (GFS).

GFS generally involves the creation of a fistula to promote the drainage of aqueous humor. Typically, this includes the creation of an elevation of the conjunctiva at the surgical site. This elevation is commonly referred to as a filtering bleb. The filtering blebs which are most often associated with good IOP control are avascular and are either low profile and diffuse, or elevated with numerous cystic spaces. Studies have suggested that aqueous fluid in the filtering bleb usually filters through the conjunctiva and mixes with the tear film, or is adsorbed by vascular or perivascular conjunctival tissue.

Although GFS is generally successful at the start in lowering the patient's IOP to normal levels, over time scar tissue forms in the filtration bleb. This reduces the drainage capacity of the filtration system created by the surgery, which then causes the patient's IOP to return to abnormally high levels [see for example: Tahery, M. M.; et. al. J. Ocul. Pharmacology, volume 5, pp. 155-179 (1989); Tripathi, R. C. Drug Dev. Res., volume 22, pp. 1-23 (1991); “Glaucoma Filtering Procedures”, in Textbook of Glaucoma (2^{nd} edition), M. Bruce Shields editor, Wilkins & Wilkins:Baltimore, 1987, chapter 34, pp. 461-487]. In order to slow the scarring process, anti-inflammatory and antiproliferative agents can be applied to the surgical site. See, e.g. U.S. Pat. No. 5,811,453, the contents of which are by this reference incorporated herein. However the use of these types of drugs has had only limited success. Many of these drugs cannot be used at the concentrations which would be most effective at suppressing scar formation since they are toxic to other ophthalmic tissue. Furthermore the surgical failure rate (from re-closure of the surgically created bleb) is high in certain groups, such as patients with previous failed filtering surgery, prior cataract extraction, aphakia, or neovascular glaucoma. Other complications seen with these agents include corneal and conjunctival epithelial loss, corneal opacification, and wound leaks due to thin blebs. Thus there exists a need for an improved drug therapy to complement GFS in order to maintain the surgically achieved enhanced outflow by preventing fistula closure due to inappropriate scarring.

3. Epothilones

The epothilones are a family of macrolactones which potently inhibit the growth of cancer cells in vitro and the growth of tumors in vivo [Danishefsky et. al., J. Org. Chem. vol. 64, p. 8434 (1999); Danishefsky et. al., Proc. Natl. Acad. Sci. USA, issue 95, p. 9643 (1998); Nicolau et. al., Angew. Chem. Intl. Ed. Engl., issue 37, p. 2014 (1998)]. They are among the most potent antitumor agents discovered to date and they maintain their efficacy against a wide range of multidrug-resistant cancers. It is believed that the ability of these compounds to inhibit inappropriate cell division is related to their ability to block the depolymerization of microtubles, and not due to direct cytotoxicity. For example, deoxyepothilone B (compound 1 below) has been shown to cause complete tumor regression in tumor-implanted mice without any noticeable toxic effects, whereas comparably effective doses of the standard anticancer agents adriamycin and paclitaxel caused some animal deaths [Danishefsky et al., J. Org. Chem. vol. 64, p. 8434 (1999)].

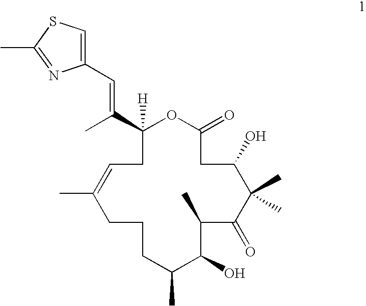

### SUMMARY OF THE INVENTION

The present invention provides improved methods to prevent and/or retard the formation of secondary cataracts and/or ophthalmic scar tissue. The methods involve the administration of an ophthalmic composition containing one or more epothilones and/or analogs thereof to the surgical site at the time of or after surgery.

### DETAILED DESCRIPTION OF THE INVENTION

The compounds of the present invention useful for the prevention or retardation of secondary cataracts and ophthalmic scarring, especially scarring associated with fistula closure after GFS are those of formula I:

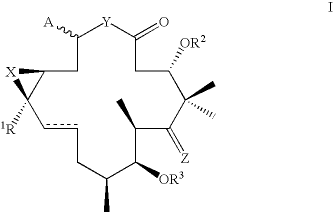

wherein:

Y=O, NH, or a functionally modified amino group;

OR² and OR³=same or different=a free or functionally modified hydroxy group;

Z=O, NOH, or NNH₂, where the OH and NH₂ may be free or functionally modified;

R¹=H or optionally substituted alkyl;

[symbol] =a single or double bond;

X is a direct bond (olefin stereochemistry in this case can be E or Z), O, CH₂, OCH₂, CH₂O, S, NH, or a functionally modified amino group;

A is E— or Z—CR⁵═CHR⁶ [the relative stereochemistry of this substituent can be α (down) or β (up)];

R⁵ is H, alkyl, or halogen; and

R⁶ is aryl, heteroaryl, cycloalkyl, cycloalkenyl, or heterocycloalkyl.

Preferred compounds of formula I are those wherein:

Y is O or NH;

R² and R³ are H;

Z is O;

R¹ is H or CH₃;

[symbol] =a single or double bond;

X is a direct bond, CH₂, O, or NH;

A is E— or Z—CR⁵═CHR⁶, the substituent being in the β (up) position (the hydrogen at the A-bearing carbon being in the α position);

R⁵ is H, C₁₋₅ alkyl, or Cl; and

R⁶ is aryl or heteroaryl.

Among the especially preferred of the foregoing compounds are the seven below:

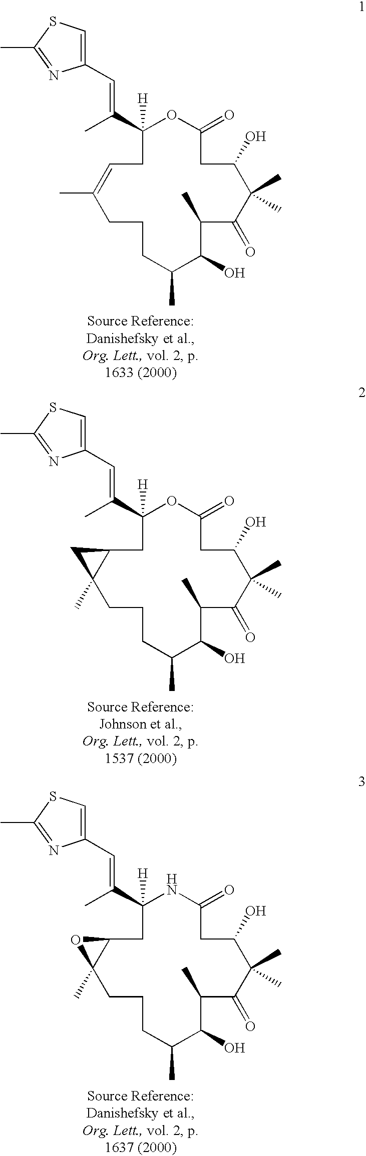

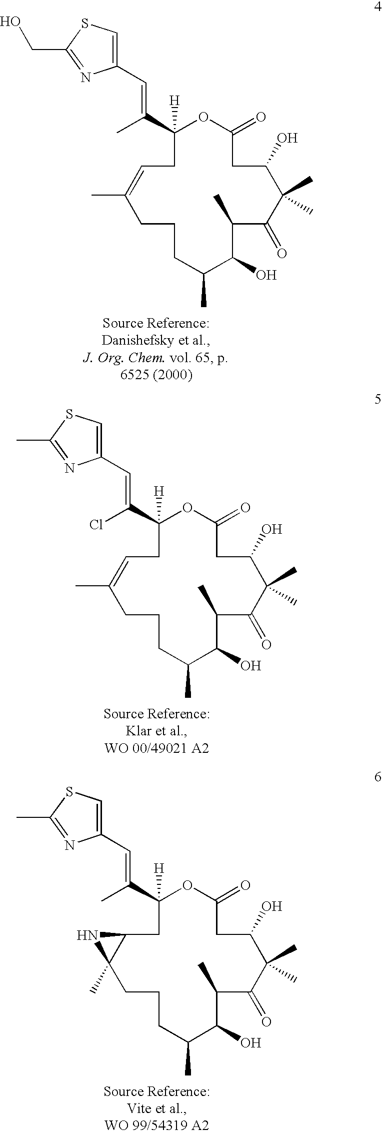

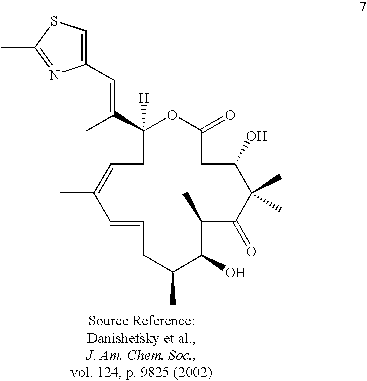

Included within the scope of the present invention are the individual enantiomers of the title compounds, as well as their racemic and non-racemic mixtures. Preferred are enantiomerically pure compositions (>98% enantiomeric excess of the desired enantiomer) with the absolute stereochemistry of the constituent molecules being as drawn for I. References for the synthesis of enantiomerically enriched samples of epothilones and analogs can be found, in addition to the epothilone-related literature cited above, in the following publications, all of which are incorporated in their entirety by reference: Johnson, J.; et al. Org. Lett., 2:1537 (2000); Danishefsky, S. J. et al. Org. Lett., volume 2, p. 1633 (2000); Danishefsky, S. J.; et al., Org. Lett., 2:1637 (2000); Danishefsky, S. J. et al., J. Am. Chem. Soc., 121:7050 (1999); Nicolaou, K. C. et al., WO 99/67252 A2; Vite, G. D. et al., WO 99/54330 A1; Danishefsky, S. J. et al., WO 99/43653 A1; Vite, G. D. et al., WO 99/54319 A1. Generally, the individual enantiomers can be procured by a number of methods, including but not limited to: enalntioselective synthesis from the appropriate enantiomerically pure or enriched starting material; synthesis from racemic/non-racemic or achiral starting materials using a chiral reagent, catalyst, solvent etc. (see for example: Asymmetric Synthesis, J. D. Morrison and J. W. Scott, Eds. Academic Press Publishers, (New York), 1985), volumes 1-5; Principles of Asymmetric Synthesis, R. E. Gawley and J. Aube, Eds.; Elsevier Publishers (Amsterdam 1996)); and isolation from racemic and non-racemic mixtures by a number of known methods, e.g. by purification of a sample by chiral HPLC (A Practical Guide to Chiral Separations by HPLC, G. Subramanian, Ed., VCH Publishers, (New York 1994); Chiral Separations by HPLC, A. M. Krstulovic, Ed., Ellis Horwood Ltd. Publishers (1989)), or by enantioselective hydrolysis of a carboxylic acid ester sample by an enzyme (Ohno, M.; Otsuka, M., Organic Reactions, 37:1 (1989)). Those skilled in the art will appreciate that racemic and non-racemic mixtures may be obtained by several means, including without limitation, nonenantioselective synthesis, partial resolution, or even mixing samples having different enantiomeric ratios. Departures may be made from such details within the scope of the accompanying claims without departing from the principles of the invention and without sacrificing its advantages. Also included within the scope of the present invention are the individual isomers substantially free of their respective enantiomers.

Wavy line attachments indicate that the configuration at that site may be either alpha (α) or beta (β). Hatched lines indicate the a configuration. A solid triangular line indicates the β configuration.

The term “free hydroxy group” means an OH. The term “functionally modified hydroxy group” means an OH which has been functionalized to form: an ether, in which an alkyl group is substituted for the hydrogen; an ester, in which an acyl group is substituted for the hydrogen; a carbamate, in which an aminocarbonyl group is substituted for the hydrogen; or a carbonate, in which an alkoxycarbonyl group is substituted for the hydrogen.

The term “free amino group” means an NH₂. The term “functionally modified amino group” means an NH₂ which has been functionalized to form: an alkoxyamino or hydroxyamino group, in which an alkoxy or hydroxy group is substituted for one of the hydrogens; an alkylamino group, in which an alkyl group is substituted for one or both of the hydrogens; an amide, in which an acyl group is substituted for one of the hydrogens; a carbamate, in which an alkoxycarbonyl group is substituted for one of the hydrogens; a urea, in which an aminocarbonyl group is substituted for one of the hydrogens; or a sulfonamide, in which an alkylsulfonyl, a cycloalkylsulfonyl, a heterocycloalkylsulfonyl, a cycloalkenylsulfonyl, an arylsulfonyl, or a heteroarylsulfonyl group is substituted for one of the hydrogens. Combinations of these substitution patterns, for example an NH₂ in which one of the hydrogens is replaced by an alkyl group and the other hydrogen is replaced by an alkoxycarbonyl group, also fall under the definition of a functionally modified amino group and are included within the scope of the present invention.

The term “sulfonyl” represents an SO₂. The sulfonyl group is bonded from the sulfur atom to two other groups. For example, a dialkylsulfonyl group can be represented as alkyl¹-SO₂-alkyl², where alkyl¹ and alkyl² are the same or different alkyl groups, which are each bonded to the sulfur atom. An arylsulfonylamide can be represented by aryl-SO₂—NWW¹, where aryl is an aryl group and NWW¹ is a free or functionally modifed amino group, with the aryl and NWW¹ groups being bonded to the sulfur atom.

The term “acyl” represents a group that is linked by a carbon atom that has a double bond to an oxygen atom and a single bond to another carbon atom.

The term “alkyl” includes straight or branched chain aliphatic hydrocarbon groups that are saturated and have 1 to 15 carbon atoms. The alkyl groups may be substituted with other groups, such as halogen, hydroxyl or alkoxy. Preferred straight or branched alkyl groups include methyl, ethyl, propyl, isopropyl, butyl and t-butyl.

The term “cycloalkyl” includes straight or branched chain saturated or unsaturated aliphatic hydrocarbon groups which connect to form one or more rings, which can be fused or isolated. The rings may be substituted with other groups, such as halogen, hydroxyl, alkoxy, or lower alkyl. Preferred cycloalkyl groups include cyclopropyl, cyclobutyl, cyclopentyl and cyclohexyl.

The term “heterocycloalkyl” refers to cycloalkyl groups which contain at least one heteroatom such as O, S, or N in the ring. Heterocycloalkenyl rings may be isolated, with 5 to 8 ring atoms, or fused, with 8 to 10 atoms. The heterocycloalkyl ring(s) hydrogens or heteroatoms with open valency may be substituted with other groups, such as lower alkyl, acyl, or halogen. Preferred heterocycloalkyl groups include piperidine, piperazine, pyrrolidine, tetrahydrofuranyl, tetrahydropyranyl, and tetrahydrothienyl.

The term “alkenyl” includes straight or branched chain hydrocarbon groups having 1 to 15 carbon atoms with at least one carbon-carbon double bond. The chain hydrogens may be substituted with other groups, such as halogen. Preferred straight or branched alkeny groups include, allyl, 1-butenyl, 1-methyl-2-propenyl and 4-pentenyl.

The term “cycloalkenyl” includes straight or branched chain, saturated or unsaturated aliphatic hydrocarbon groups which connect to form one or more non-aromatic rings containing a carbon-carbon double bond, which can be fused or isolated. The rings may be substituted with other groups, such as halogen, hydroxyl, alkoxy, or lower alkyl. Preferred cycloalkenyl groups include cyclopentenyl and cyclohexenyl.

The term “alkoxy” represents an alkyl group attached through an oxygen linkage.

The term “carbonyl group” represents a carbon atom double bonded to an oxygen atom, wherein the carbon atom has two free valencies.

The term “alkoxycarbonyl” represents an alkoxy group bonded from its oxygen atom to the carbon of a carbonyl group, the carbonyl group itself being bonded to another atom through its carbon atom.

The term “aminocarbonyl” represents an amino group bonded from its nitrogen atom to the carbon atom of a carbonyl group, the carbonyl group itself being bonded to another atom through its carbon atom.

The term “lower alkyl” represents alkyl groups containing one to six carbons (C₁—C₆).

The term “halogen” represents fluoro, chloro, bromo, or iodo.

The term “aryl” refers to carbon-based rings which are aromatic. The rings may be isolated, such as phenyl, or fused, such as naphthyl. The ring hydrogens may be substituted with other groups, such as lower alkyl, or halogen.

The term “heteroaryl” refers to aromatic hydrocarbon rings which contain at least one heteroatom such as O, S, or N in the ring. Heteroaryl rings may be isolated, with 5 to 6 ring atoms, or fused, with 8 to 10 atoms. The heteroaryl ring(s) hydrogens or heteroatoms with open valency may be substituted with other groups, such as lower alkyl or halogen. Examples of heteroaryl groups include imidazole, pyridine, indole, quinoline, furan, thiophene, pyrrole, tetrahydroquinoline, dihydrobenzofuran, and dihydrobenzindole.

The compounds of this invention may be administered by any of several routes, including: a) topical ocular, intraocular injection, or irrigating solution delivery; b) application from a surgical sponge; or c) incorporation into an implantable film, viscoelastic, or other bioerodible device, which is left in the eye for sustained delivery to the affected area.

Generally topical ophthalmic compositions are preferred for preventing or retarding fistula closure, and will be in the form of a solution, suspension, gel, or formulated as part of a device, such as a collagen shield or other bioerodible or non-bioerodible device. Various excipients may be contained in the topical ophthalmic solutions, suspensions or gels of the present invention. For example, buffers (e.g., borate, carbonate, phosphate), tonicity agents (e.g., sodium chloride, potassium chloride, polyols), preservatives (e.g., polyquaterniums, polybiguanides, BAC), chelating agents (e.g., EDTA), viscosity enhancing agents (e.g., polyethoxylated glycols) and solubilizing agents (e.g., polyethoxylated castor oils, including polyoxl-35 castor oil (Cremophor EL®, BASF Corp., Parsippany, N.J.); Polysorbate 20, 60 and 80; Pluronic® F-68, F-84 and P-103 (BASF Corp.); or cyclodextrin) may be included in the topical ophthalmic compositions. A variety of viscoelastics and gels may be useful in topical ophthalmic gel compositions of the present invention, including, but not limited to, mucopolysaccharides (e.g. hyaluronates and chondroitin sulfates) carbomers, polyvinyl alcohol-borate complexes, or xanthan, gellan, or guar gums.

Non-solid vehicles useful for delivery of the compounds of the present invention are preferably aqueous and are formulated so as to be chemically and physically compatible with ophthalmic tissues. For example, viscoelastic formulations currently utilized in connection with intraocular surgical procedures, such as HEALON® (sodium hyaluronate) (Pharmacia Corp., Peapack, N.J., USA), PROVISC® (sodium hyaluronate) (Alcon Laboratories, Inc., Fort Worth, Tex., USA), or VISCOAT® (sodium chondroitin sulfate-sodium hyaluronate) (Alcon Laboratories, Inc.), or CELLUGEL® (hydroxy-propylmethyl-cellulose) are preferred as vehicles for the above-described compounds, especially for the prevention or retardation of secondary cataracts. Such viscous formulations tend to adhere to tissue.

Preferred among such viscous formulations for use in preventing or retarding secondary cataract formation are those containing hyaluronic acid or its ophthalmically acceptable salts, esters and amides, which are relatively more dispersive, i.e. less cohesive, than HEALON®, such as VISCOAT® or the hyaluronic acid amide derivatives disclosed in WO 00/01733, the contents of which are by this reference incorporated herein. Most preferred among the viscoelastic vehicles are “non-aspirating” viscoelastics that may be left in the eye at the close of surgery without significant risk of a deleterious, post-operative intraocular pressure spike. Examples of such non-aspirating viscoelastics, including hydrophobically modified hyaluronate and especially hyaluronic acid (HA) amides, are disclosed in commonly assigned U.S. patent application Ser. Nos. 10/111,524 and 10/111,520 the contents of which are by this reference incorporated herein. These properties help to ensure that the compositions will expose lens epithelial cells to the actions of the epothilones of the present invention. This is particularly true when the compositions are applied following removal of the natural crystallin lens, since at that point the capsular bag will be at least partially open and therefore prone to immediately losing any fluid which is applied to the interior of the bag by means of irrigation. The use of a viscous solution or semi-solid composition may therefore be preferable in some cases. In other cases, such as those where the epothilone composition is injected into the capsular bag prior to removal of the natural crystallin lens, the viscosity of the composition will not be a primary concern, since leakage of the composition from the capsular bag will be a less significant problem. The use of a less viscous aqueous solution as the vehicle may therefore be preferred in such cases.

The above-described compositions can be applied to the lens capsule by means of various techniques. For example, the compositions can be applied to the interior of the capsular bag by means of a syringe following removal of the crystallin lens, or can be injected into the lens capsule prior to removal of the crystallin lens by means of phacoemulsification or other methods.

The less viscous aqueous solutions useful as a vehicle for the compounds of the present invention, whether to reduce secondary cataract formation or fistula closure, must similarly be compatible with intraocular tissues, and should preferably help to maintain the integrity and function of intraocular tissues during or after the surgical procedure. The most basic irrigating solutions generally comprise saline, or phosphate-buffered saline. More advanced irrigating solutions, however, are preferred. As used herein, the term “physiologically balanced irrigating solution” refers to a solution which is adapted to maintain the physical structure and function of tissues during invasive or noninvasive medical procedures. This type of solution will typically contain electrolytes, such as sodium, potassium, calcium, magnesium and/or chloride; an energy source, such as dextrose; and a bicarbonate-buffer to maintain the pH of the solution at or near physiological levels. Various solutions of this type are known (e.g., Lactated Ringers Solution), BSS® Sterile Irrigating Solution, and BSS Plus® Sterile Intraocular Irrigating Solution (Alcon Laboratories, Inc., Fort Worth, Tex., USA) are examples of physiologically balanced intraocular irrigating solutions. The latter type of solution is described in U.S. Pat. No. 4,550,022 (Garabedian, et al.), the entire contents of which are incorporated herein by reference. Preferred aqueous solutions include physiologically balanced irrigating solutions, such as BSS® (Balanced Salt Solution) and BSS Plus® (Balanced Salt Solution Enriched with Bicarbonate, Dextrose and Glutathione) available from Alcon Laboratories, Inc., Fort Worth, Tex. Also preferred is BION TEARS® Lubricant Eye Drops, also available from Alcon Laboratories, Inc., Fort Worth, Tex.

As will be appreciated by those skilled in the art, the above-described compositions must be sterile and should not include any agents (e.g., antimicrobial preservatives) which will be toxic to sensitive ocular tissues. Preservatives can be used to prevent microbial contamination during use. Suitable preservatives include: benzalkonium chloride, thimerosal, chlorobutanol, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% weight/volume (“% w/v”). The above-described compositions can be formulated in accordance with techniques known to those skilled in the art.

The epothilones of the invention can also be incorporated in an intraocular drug delivery system providing a slower release effect than would ordinarily be achieved with the previously discussed aqueous and viscoelastic vehicles. Topical ophthalmic bioerodible and non-bioerodible devices (e.g., conjunctival implant) are known in the art and may be useful in the topical administration of formula (I) compounds. See, for example, Weiner, A. L., Polymeric Drug Delivery Systems for the Eye, in Polymeric Site-specific Pharmacotherapy, Ed., A. J. Domb, John Wiley & Sons, pages 316-327 (1994). While the particular ingredients and amounts to be contained in topical ophthalmic compositions useful in the methods of the present invention will vary, particular topical ophthalmic compositions will be formulated to effect the administration of a compound of formula (I) topically to the eye. The active substance in solid form could be coated with or encapsulated by an ophthalmologically acceptable carrier substance. Examples of systems, which are generally known for encapsulating drugs, are liposomes which are membrane-like vesicles, and microspheres based on polymers of lactic and glycolic acid. A slow release system can alternatively be prepared by adding the epothilone of interest in dissolved form to a carrier matrix under conditions so that a desired amount of the substance is incorporated. An example of such a carrier matrix is a gel, for instance a biodegradable gel of hyaluronic acid as disclosed in EP 408731. Additionally, a slow release composition comprising the epothilone of interest can be deposited directly on the tissue at the surgical site, under conditions so that the composition is bound to the tissue or forms an interpenetrating network with the tissue surface layer.

A preferred embodiment of the invention comprises intraocular administration of the epothilone compound in an amount of about 0.005 to 5 μg and especially 0.1 to 5 μg, in approximately 0.1 mL of a viscoelastic medium, especially PROVISC®, Healon® or Viscoat® sterile ophthalmic viscoelastic solutions. In slow release systems, the dose would preferably be considerably higher, for instance up to about 25 μg, concentration being dictated by the volume of the vehicle or implant. Another preferred embodiment comprises irrigating solution delivery of the epothilone compound at a concentration of about 0.05 to about 100 μg per mL.

Representative preferred formulations are described in the following Examples.

### EXAMPLE 1

| Ingredient | % w/v |
|---|---|
| Compound of formula I | 0.000023 |
| Cremophor EL | 0.05 |
| Hyaluronic Acid, Sodium Salt | 1 |
| Dibasic Sodium Phosphate (Anhydrous) | 0.056 |
| Monobasic Sodium Phosphate (Monohydrate) | 0.004 |
| Sodium Chloride | 0.84 |
| Hydrochloric Acid | pH adjusted |
| Sodium Hydroxide | pH adjusted |
| Water | QS |

### EXAMPLE 2

| Ingredient | % w/v |
|---|---|
| Compound 1 | 0.00001-0.0010 |
| Cremophor EL | 0.05 |
| Sodium Chondroitin Sulfate | 4.0 |
| Sodium Hyaluronate | 3.0 |
| Sodium Dihydrogen Phosphate, Monohydrate | 0.045 |
| Disodium Hydrogen Phosphate, Anhydrous | 0.2 |
| Sodium Chloride | 0.310 |
| Water | QS |
| Hydrochloric Acid | pH adjusted |
| Sodium Hydroxide | pH adjusted |

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

What is claimed is:

1. A method of reducing the formation of scar tissue or posterior capsular pacification in an eye following surgery thereon, comprising administering to the affected eye an ophthalmic composition containing a therapeutically effective amount of one or more epothilones of formula I: 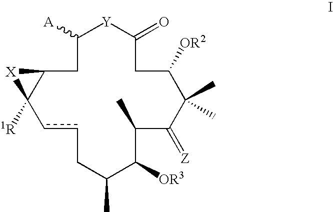
wherein:
Y=O NH, or a functionally modified amino group;
OR² and OR³=same or different=a free or functionally modified hydroxy group;
Z=O, NOH, or NNH₂, where the OH and NH₂ may be free or functionally modified;
R¹=H or optionally substituted alkyl; [symbol]
=a single or double bond;
X is a direct bond (olefin stereochemistry in this case can be E or Z), O, CH₂, OCH₂, CH₂O, S, NH, or a functionally modified amino group;
A is E— or Z—CR⁵═CHR⁶;
R⁵ is H, alkyl, or halogen; and
R⁶ is is aryl, heteroaryl, cycloalkyl, cycloalkenyl, or heterocycloalkyl.

2. The method of claim 1, wherein for the compound(s) of formula I:
Y is O or NH,
R² and R³ are H;
Z is O;
R¹ is H or CH₃; [symbol]
=a single or double bond
X is a direct bond, CH₂, O, or NH;
A is E— or Z—CR⁵═CHR⁶ in the β position;
R⁵ is H, C₁₋₅ alkyl, or Cl; and
R⁶ is aryl or heteroaryl.

3. The method of claim 2, wherein the compound(s) of formula I is(are) selected from the group consisting of:

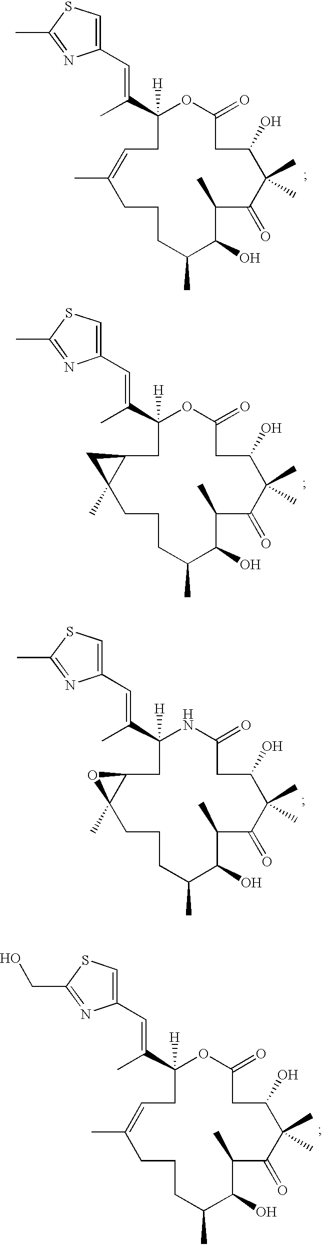

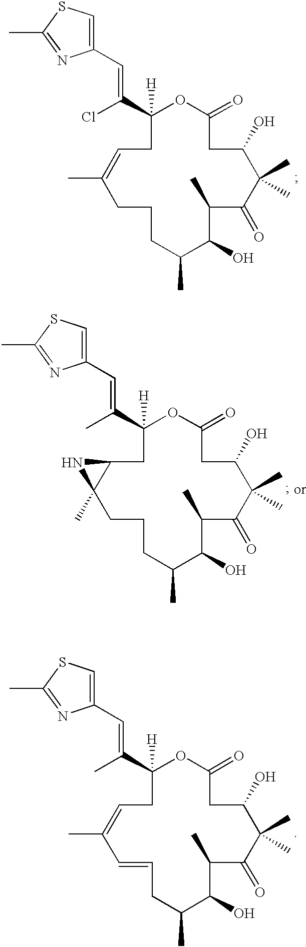

4. The method according to any of claims 1-3, wherein the composition is administered during or after cataract surgery.

5. The method according to any of claims 1-3, wherein the ophthalmic composition is administered during or after glaucoma filtration surgery.

6. The method of claim 5, wherein the composition is administered to reduce the formation of scar tissue in a filtration bleb or drainage fistula of the eye.

7. A method according to claim 5, wherein the composition is administered to the surgical site via topical application of a pre-soaked sponge containing the composition.

8. A method according to any of claims 1-5, wherein the composition is administered to the eye in a surgical irrigating solution.

9. A method according to any of claims 1-5, wherein the composition is administered in an ophthalmically acceptable viscoelastic medium.

10. A method according to claim 9, wherein the viscoelastic medium comprises an aqueous solution of hyaluronic acid.

11. A method as defined by according to any of claims 1-5, wherein the compound(s) of formula I are administered using a slow release composition.
